(19) United States
(12) Patent Application Publication
(10) Pub. No.: US 2022/0149476 A1
(43) Pub. Date: May 12, 2022

(54) ELECTRICAL ENERGY STORE, DEVICE AND METHOD FOR OPERATING AN ELECTRICAL ENERGY STORE

(71) Applicant: Robert Bosch GmbH, Stuttgart (DE)

(72) Inventor: Johannes Schneider, Stuttgart (DE)

(21) Appl. No.: 17/509,320

(22) Filed: Oct. 25, 2021

(30) Foreign Application Priority Data
Nov. 6, 2020 (DE) 10 2020 213 990.3

(51) Int. Cl. H01M 50/30; G01N 33/00

(52) U.S. Cl. CPC H01M 50/30; G01N 33/0027

(57) ABSTRACT

Electrical energy store (1) and method (100) for operating an electrical energy store (1) having at least one electrical energy storage cell (5), a printed circuit board (2) and a gas sensor (4), wherein each electrical energy storage cell (5) has a respective degassing opening (3),

wherein the gas sensor (4) is arranged between a respective degassing opening (3) and the printed circuit board (2).

### FIELD OF THE INVENTION

The present invention relates to an electrical energy store, a device and a method for operating an electrical energy store.

DE 10 2014 202 635 A1 discloses a battery cell having a power interruption during degassing.

U.S. Pat. No. 9,660,237 B2 discloses a multi-compartment ventilation duct for a battery module.

### SUMMARY OF THE INVENTION

Embodiments described herein provide an electrical energy store, having at least one electrical energy storage cell, a printed circuit board and a gas sensor, wherein a respective electrical energy storage cell has a respective degassing opening, is that the gas sensor is arranged between the respective degassing opening and the printed circuit board.

The electrical energy store can have a compact design by the printed circuit board being able to be arranged with the control unit of the electrical energy store above the electrical energy storage cell in a housing of the electrical energy store. In this case, the housing of the electrical energy store surrounds the electrical energy storage cells, the printed circuit board and the gas sensor so as to form a housing.

The arrangement of the gas sensor between the degassing openings and the printed circuit board means that a gas stream passing out of a degassing opening impinges first on the gas sensor and then on the printed circuit board. As a result, the gas sensor identifies the gas stream first before the printed circuit board can be damaged by the gas stream.

Further advantageous embodiments of the present invention are the subject of the dependent claims.

Advantageously, the electrical energy store has a plurality of electrical energy storage cells and a single gas sensor, wherein the gas sensor is arranged between the degassing openings of all the electrical energy storage cells and the printed circuit board.

According to one advantageous configuration, the gas sensor is designed as an electrical conductor, in particular as a wire, in particular wherein the gas sensor extends along the degassing openings. A gas stream passing out of a degassing opening can therefore be detected in a simple manner.

Advantageously, the electrical conductor is designed to be so sensitive that the gas sensor can detect the gas stream as early as upon first contact with the gas stream.

It is advantageous in this case for the electrical energy storage cells to be arranged next to one another in such a way that the degassing openings are arranged along a line, in particular along a straight line. The gas sensor can therefore be of linear design.

It is also advantageous for the gas sensor to be configured to be severed if a limit temperature is exceeded or in the case of contamination with gases from the interior of an electrical energy storage cell. A hot gas stream that arises in the case of a defect in an electrical energy storage cell can therefore be detected in a simple manner.

According to a further advantageous configuration, the gas sensor is configured to change its electrical resistance if a limit temperature is exceeded or in the case of contamination with gases from an electrical energy storage cell. The gas sensor can therefore be evaluated in a simple manner.

Advantageously, the gas sensor is connected to the electrical energy storage cells and/or the printed circuit board by means of connecting elements, in particular wherein the gas sensor is spaced apart from the printed circuit board and/or the electrical energy storage cells by means of the connecting elements. It is therefore possible to avoid inadvertent triggering of the gas sensor.

It is also advantageous for electronic components of a control unit of the electrical energy store to be arranged on the printed circuit board. In this case, the control unit can be integrated into the housing of the electrical energy store and the electrical energy store can have a compact design.

It is advantageous in this case for a carrier material of the printed circuit board to be arranged between the electronic components and the gas sensor. As a result, the electronic components are protected from the gas stream by means of the carrier material.

Some embodiments provide a device, in particular a vehicle, that has an electrical energy store as described above or according to one of the claims relating to the electrical energy store.

It is possible to detect degassing of an electrical energy storage cell early so that a user of the device can be informed and accordingly can switch off the device and distance themselves from the device before the electrical energy store becomes ignited, for example.

Some embodiments provide a method for operating an electrical energy store as described above or according to one of the claims relating to the electrical energy store is that, in a first method step, a gas sensor indicates an escape of gas from an electrical energy storage cell of the electrical energy store, wherein, in a second method step, the electrical energy store sends a message to a superordinate controller that the gas sensor has indicated an escape of gas, wherein, in a third method step, there is a waiting period to check whether a control unit of the electrical energy store fails within a defined period, wherein, in a fourth method step, a safety action is executed if the gas sensor has indicated an escape of gas and the control unit has failed within the defined period.

In some embodiments, the gas sensor can be designed to be simple and sensitive and unnecessary safety actions can be avoided in spite of this. As a result of the fact that neither the notification from the gas sensor alone, nor the failure of the control unit alone, leads to a safety action, both a faulty gas sensor and any other disturbance of the control unit can be ruled out before a safety action is executed.

The above configurations and developments can be combined with one another as desired, where appropriate. Further possible configurations, developments and implementations of the invention also comprise combinations that are not explicitly mentioned of features of the invention that are described above or below with respect to the exemplary embodiments. In particular, a person skilled in the art will in this case also add individual aspects as improvements or additions to the respective basic form of the present invention.

### BRIEF DESCRIPTION OF THE DRAWINGS

In the section that follows, the invention is explained on the basis of exemplary embodiments, from which further inventive features can arise, but to which the scope of the invention is not restricted. The exemplary embodiments are illustrated in the drawings.

In the figures:

FIG. 1 shows an electrical energy store 1 according to the invention in a partially transparent plan view,

FIG. 2 shows the electrical energy store 1 according to the invention in a side view, and

FIG. 3 shows a method 100 according to the invention for operating an electrical energy store 1.

### DETAILED DESCRIPTION

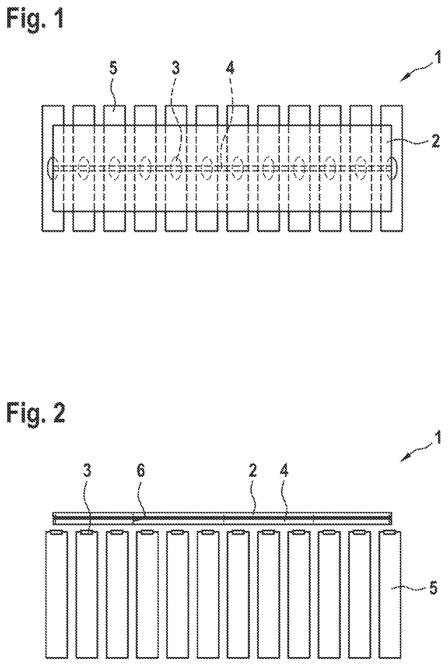

The electrical energy store 1 according to the invention illustrated in FIG. 1 and FIG. 2 has electrical energy storage cells 5, a printed circuit board 2 and a gas sensor 4.

Each electrical energy storage cell 5 has a degassing opening 3.

The electrical energy storage cells 5 are arranged next to one another in such a way that the degassing openings 3 are arranged along a line, in particular a straight line.

The printed circuit board 2 is arranged above the electrical energy storage cells 5. In this case, the degassing openings 3 are arranged in a respective side wall of the respective electrical energy storage cell 5 that is facing the printed circuit board 2.

The gas sensor 4 is arranged between the printed circuit board 2 and the electrical energy storage cells 5. In this case, the gas sensor 4 extends along the degassing openings 3. The gas sensor 4 is preferably designed as an electrical conductor, in particular a wire, that is configured to change its electrical resistance and/or to be severed if a limit temperature is exceeded or in the case of contamination with gases from the interior of an electrical energy storage cell 5. In this case, the gas sensor 4 can melt through or become brittle and tear.

The gas sensor 4 is connected to the electrical energy storage cells 5 and/or the printed circuit board 2 by means of connecting elements 6. The gas sensor 4 is arranged so as to be spaced apart from the electrical energy storage cells 5 and the printed circuit board 2, in particular wherein the connecting elements 6 are arranged between the gas sensor 4 and the printed circuit board 2 and/or the electrical energy storage cells 5. The number of connecting elements 6 is smaller than the number of electrical energy storage cells 5. The connecting elements 6 have a material thickness that is smaller than 3 mm, in particular smaller than 1 mm.

Electronic components of a control unit of the electrical energy store 1 are arranged on the printed circuit board 2. In this case, a carrier material of the printed circuit board 2 is arranged between the gas sensor 4 and the electronic components.

According to an alternative embodiment that is not illustrated in the figures, the gas sensor 4 can be designed as a light barrier that is configured to detect gas flowing out of an electrical energy storage cell 5.

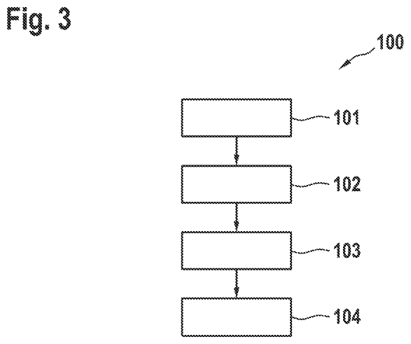

The method 100 illustrated in FIG. 3 for operating an electrical energy store 1 has the following temporally successive method steps:

In a first method step 101, a gas sensor 4 indicates an escape of gas from an electrical energy storage cell 5 of the electrical energy store 1.

In a second method step 102, the electrical energy store 1 sends a message to a superordinate controller that the gas sensor 4 has indicated an escape of gas.

In a third method step 103, there is a waiting period to check whether a control unit of the electrical energy store 1 fails within a defined period.

In a fourth method step 104, a safety action is executed if the gas sensor 4 has indicated an escape of gas and the control unit has failed within the defined period. As the safety action, a warning signal can sound or be displayed, for example vehicle occupants can be requested to stop and leave a vehicle having the electrical energy store 1.

The defined period here is less than 30 s, in particular less than 20 s, preferably less than 10 s. It is chosen depending on the dimensions of the electrical energy store 1 such that it is long enough for the gas stream, once the gas sensor 4 has indicated it, to cause failure of the control unit. On the other hand, the defined period has to be chosen to be short enough so that it is possible to avoid accidental correlation of the indication of the gas sensor 4 and the failure of the control unit.

In this context, an electrical energy store is understood to mean a rechargeable energy store, in particular having an electrochemical energy storage cell and/or an energy storage module having at least one electrochemical energy storage cell and/or an energy storage pack having at least one energy storage module. The energy storage cell can be designed as a lithium-based battery cell, in particular a lithium-ion battery cell or a lithium-iron-phosphate battery cell. Alternatively, the energy storage cell is designed as a lithium-polymer battery cell or nickel-metal hydride battery cell or lead-acid battery cell or lithium-air battery cell or lithium-sulfur battery cell.

In this context, a vehicle is understood to mean a land vehicle, for example an automobile or a truck or a two-wheeled vehicle, or an aircraft or a watercraft. The vehicle is, for example, understood to be an at least partly electrically driven vehicle, in particular an electric battery-driven vehicle that has a purely electrical drive, or a hybrid vehicle that has an electrical drive and an internal combustion engine.

1. An electrical energy store (1) comprising:
at least one electrical energy storage cell (5);
a printed circuit board (2); and
a gas sensor (4),
wherein the at least one electrical energy storage cell (5) has a degassing opening (3), and
wherein
the gas sensor (4) is arranged between the degassing opening (3) and the printed circuit board (2).

2. The electrical energy store (1) according to claim 1,
wherein the at least one electrical energy storage cell (5) includes a plurality of electrical energy storage cells (5), wherein each of the plurality of electrical energy storage cells (5) has a respective degassing opening (3), and wherein the gas sensor (4) includes a single sensor and is arranged between the degassing openings (3) of all the plurality of electrical energy storage cells (5) and the printed circuit board (2).

3. The electrical energy store (1) according to claim 2,
wherein
the gas sensor (4) includes an electrical conductor extending along the degassing openings (3) of all of the plurality of electrical energy storage cells (5).

4. The electrical energy store (1) according to claim 1,
wherein
the plurality of electrical energy storage cells (5) are arranged next to one another in such a way that the degassing openings (3) of each of the plurality of electrical energy storage cells (5) are arranged along a straight line.

5. The electrical energy store (1) according to claim 1,
wherein
the gas sensor (4) is configured to be severed if a limit temperature is exceeded or in the case of contamination with gases from an interior of the at least one electrical energy storage cell (5).

6. The electrical energy store (1) according to claim 1,
wherein
the gas sensor (4) is configured to change its electrical resistance if a limit temperature is exceeded or in the case of contamination with gases from the at least one electrical energy storage cell (5).

7. The electrical energy store (1) according to claim 1,
wherein
the gas sensor (4) is connected to at least one of the at least one electrical energy storage cell (5) and the printed circuit board (2) via a connecting element (6), wherein the gas sensor (4) is spaced apart from at least one of the printed circuit board (2) and the at least one electrical energy storage cell (5) by the connecting element (6).

8. The electrical energy store (1) according to claim 1,
wherein
electronic components of a control unit of the electrical energy store (1) are arranged on the printed circuit board (2).

9. The electrical energy store (1) according to claim 8,
wherein
a carrier material of the printed circuit board (2) is arranged between the electronic components and the gas sensor (4).

10. A vehicle comprising:
an electrical energy store (1) including:
at least one electrical energy storage cell (5);
a printed circuit board (2); and
a gas sensor (4),
wherein the at least one electrical energy storage cell (5) has a degassing opening (3), and
wherein the gas sensor (4) is arranged between the degassing opening (3) and the printed circuit board (2).

11. A method (100) for operating an electrical energy store (1), the method comprising:
receiving, from a gas sensor (4) included in the electrical energy store (1), an indication of an escape of gas from an electrical energy storage cell (5) of the electrical energy store (1),
sending a message to a superordinate controller that the gas sensor (4) has indicated an escape of gas,
checking, during a waiting period, whether a control unit of the electrical energy store (1) fails within a defined period, and
executing a safety action if the gas sensor (4) has indicated an escape of gas and the control unit has failed within the defined period.
